(19) United States
(12) Patent Application Publication
(10) Pub. No.: US 2026/0021089 A1
(43) Pub. Date: Jan. 22, 2026

(54) APPLICATION METHOD OF BRUCINE FOR TREATING AMYOTROPHIC LATERAL SCLEROSIS

(71) Applicant: Zhejiang Chinese Medical University, Hangzhou (CN)

(72) Inventors: Chengping Wen, Hangzhou (CN); Mingzhu Wang, Hangzhou (CN); Qianqian He, Hangzhou (CN); Xiaofei Zhou, Hangzhou (CN); Tengyue Wang, Hangzhou (CN)

(21) Appl. No.: 19/226,126

(22) Filed: Jun. 2, 2025

(30) Foreign Application Priority Data
Jul. 19, 2024 (CN) 202410974825.7

(51) Int. Cl. A61K 31/475; A61P 25/28

(52) U.S. Cl. CPC A61K 31/475; A61P 25/28

(57) ABSTRACT

An application method of brucine for treating amyotrophic lateral sclerosis (ALS) is provided, relating to the field of biomedicines. It can delay a time of onset of ALS, prolong survival time of ALS patients, delay a weakening of limb extension ability, and improve limb grip strength.

### CROSS-REFERENCE TO RELATED APPLICATION

This application claims priority to Chinese patent application No. 202410974825.7, filed to China National Intellectual Property Administration (CNIPA) on Jul. 19, 2024, which is herein incorporated by reference in its entirety.

### TECHNICAL FIELD

The disclosure relates to the technical field of biomedicines, and more particularly to an application method of brucine for treating amyotrophic lateral sclerosis.

### BACKGROUND

Amyotrophic lateral sclerosis (ALS) is a progressive, fatal neurodegenerative disease primarily affecting motor neurons, which is a predominant subtype of motor neuron disease (MND), accounting for 80% to 90% of MND cases and often representing a terminal stage of other MND subtypes. ALS is a rare disease, which has a global incidence of approximately 3 per 100,000 individuals, with an average onset age of 55 to 60 years old and an average survival period of 3 years to 5 years. Most patients die due to respiratory muscle failure. ALS is categorized into familial amyotrophic lateral sclerosis (fALS) and sporadic amyotrophic lateral sclerosis (sALS), an incidence rate of fALS accounts for about 5% to 10% of a total ALS, and a time of onset of fALS is often earlier than that of sALS. 20% to 25% of fALS and 3% of sALS are related to genetic mutations in superoxide dismutase 1 (SOD1).

ALS, a refractory neurological disease, is characterized by chronic progressive degeneration of ventricornu, brainstem posterior motor nuclei, and pyramidal tracts, with simultaneous upper and lower motor neurons being affected. The pathogenesis of ALS remains inconclusive to date. Currently, the treatment of ALS patients is primarily based on symptomatic therapy, with no effective cure available. The U.S. Food and Drug Administration (FDA) has approved drugs of Riluzole and Edaravone for ALS patients, which can delay disease progression to some extent. However, their clinical application is limited due to high costs and numerous side effects. The long-term use of Western medicine and its associated side effects has prompted people to re-examine traditional therapies from a new perspective, in the hope of finding new treatment methods.

Traditional chinese medicine demonstrates therapeutic effects through multi-pathway and multi-target mechanisms, offering unique advantages in disease treatment. The traditional chinese medicine of Nux vomica seed is the dried ripe seed of Strychnos nux-vomica L. (Fam. Loganiaceae), which includes properties of activating collaterals and alleviating pain and dispersing nodules and reducing swelling. Ancient and modern medical experts have accumulated rich experience in its clinical application, modern pharmacological research and clinical practice have shown that the Nux vomica seed exhibits a wide range of pharmacological activities, such as anti-inflammatory, analgesic, anticoagulant and other pharmacological effects. In addition, it can also promote fracture healing and treat orthopedic diseases such as bone hyperplasia. However, due to high toxicity of the Nux vomica seed and narrow therapeutic window of ALS, the clinical application of the Nux vomica seed is limited. The 2020 edition of the Chinese Pharmacopocia stipulates a dosage of 0.3 grams (g)-0.6 g for the Nux vomica seed. It has been reported in the literature that toxicity reactions may occur when the dosage exceeds 0.9 g, and an oral intake of 7 g of raw Nux vomica seed can be fatal, with acute onset of symptoms. Therefore, how to achieve the goal of reducing toxicity and enhancing efficacy of the Nux vomica seed through various means is an important direction for current research. The key to solving this problem is to control the “amount” of its toxic components. Brucine, an active component of the Nux vomica seed, has shown unique advantages in this regard. Compared with the Nux vomica seed, brucine has lower toxicity, a longer half-life, and higher bioavailability. Research by scholars both domestically and internationally has proven that brucine has significant effects such as analgesia, anti-inflammation, anti-tumor, and central nervous system excitation. However, a potential efficacy of brucine in the treatment of ALS has not yet been studied.

### SUMMARY

A purpose of the disclosure is to solve the problems in the related art and propose an application methond of brucine for treating amyotrophic lateral sclerosis (ALS), which delay a time of onset of ALS, prolong survival time of ALS patients, delay a weakening of limb extension ability, and improve limb grip strength.

To achieve the above purpose, an application method of brucine for treating ALS is provided.

In an embodiment, the ALS is a progressive, fatal neurodegenerative disease affecting motor neurons.

In an embodiment, the ALS is a predominant subtype of motor neuron disease (MND) and accounts for 80% to 90% of MND cases.

In an embodiment, the ALS is a mammalian ALS.

In an embodiment, the treating ALS by using the brucine includes: delaying a time of onset of the ALS and prolonging survival time of ALS patients.

In an embodiment, the treating ALS by using the brucine includes: delaying a weakening of limb extension ability.

In an embodiment, the treating ALS by using the brucine includes: improving limb grip strength.

In an embodiment, a dosage form of the brucine is one selected from the group consisting of tablets, capsules, granules, powders, pills, lozenges, dustpowder, solutions, syrups, suspensions, emulsions, and elixirs.

In an embodiment, the treating ALS by using the brucine includes: simultaneously administering the brucine for treating the ALS and other drugs for treating the ALS.

In an embodiment, the other drugs for treating the ALS includes riluzole and edaravone.

The beneficial effects of the disclosure are as follows.

The disclosure provides the application method of brucine for treating ALS, discovering a new use for the brucine. This expands the medical applications of brucine and offers a new technological method for treating the ALS. The brucine has been shown to delay the onset of ALS, prolong survival time of the ALS patients, delay the weakening of limb extension ability, and improve limb grip strength.

The features and advantages of the disclosure will be described in detail through embodiments combined with the attached drawings.

### BRIEF DESCRIPTION OF DRAWINGS

FIG. 1 illustrates a weight change diagram of mice treated with an application method of brucine for treating amyotrophic lateral sclerosis (ALS) in the disclosure.

FIG. 2 illustrates a time of onset diagram of the mice treated with the application method of the brucine for treating ALS in the disclosure.

FIG. 3 illustrates a survival curve of the mice treated with the application method of the brucine for treating ALS in the disclosure.

FIGS. 4A to 4C illustrate hind-limb extension images of the mice treated with the application method of the brucine for treating ALS as a disease progresses in the disclosure.

FIG. 5 illustrates a grip strength diagram of the mice treated with the application method of the brucine for treating ALS in the disclosure.

FIGS. 6A to 6E illustrate maximum motor capacity diagrams of the mice treated with the application method of the brucine for treating ALS in the disclosure.

### DETAILED DESCRIPTION OF EMBODIMENTS

Human superoxide dismutase 1 with a glycine-to-alanine substitution at position 93 (hSOD1^{G93A}) transgenic mice are selected as ALS model mice. The 93^{rd} alanine residue of hSOD1 is replaced by glycine (G93A) in the mice, resulting in the inability to scavenge superoxide radicals in the body, excessive production of reactive oxygen species (ROS), and oxidative damage to the body. The ALS model mice are a classic model for studying ALS. The application effects of brucine in ALS are evaluated through the classic model.

Based on the acute toxicity experiments of the brucine conducted previously, it has been determined that when a drug dosage is less than or equal to 40 milligrams per kilogram (mg/kg), no mortality is observed in the treated mice, and no signs of poisoning are exhibited. This dosage range is considered safe. Considering that toxicity of the brucine may accumulate with long-term gavage administration, in order to ensure the safety of mice undergoing long-term gavage administration, a dosage of 10 mg/kg is determined for the efficacy trial of brucine in treating the ALS.

The efficacy of the brucine in treating the ALS animial models (the ALS model mice) of hSOD1^{G93A} transgenic mice are evaluated by gavage administration of 10 mg/kg of brucine. A motor function of the ALS model mice is measured, and a time of onset and survival period of the mice are recorded to provide preliminary experimental evidence for the efficacy and mechanism of action of brucine in treating ALS animal models.

### 2.1 Animal Grouping and Administration

Ten 8-week-old hSOD1^{G93A} transgenic mice are selected and randomly divided into a model group and a treated group according to their numerical numbers, five hSOD1^{G93A} transgenic mice in each of the model group and the treated group. Five wild-type mice from the same litter are allocated to a normal control group. The hSOD1^{G93A} transgenic mice in the model group are administered 10 mg/kg of 0.9% saline by gavage administration. The hSOD1^{G93A} transgenic mice in the treated group are administered 10 mg/kg of brucine by gavage administration. The wild-type mice in the normal control group are administered 10 mg/kg of 0.9% saline by gavage administration.

### 2.2 Weight Changes

Weights of each group of mice are measured and recorded every week by using electronic scales.

### 2.3 Time of Onset and Survival Period

### 2.3.1 Time of Onset

The time of onset of disease is assessed daily using a Weydt 4-point scale (as shown in Table 2-1). An initial time when a Weydt score is less than 4 points is recorded as the time of onset of disease.

### 2.3.2 Survival Period

The mice are placed in a supine position and if they fail to turn over to a prone position within 30 seconds, which is defined as a Weydt score of 0, this is recorded as a time of death.

TABLE 2-1
Weydt 4-point scale

| Score | Behavioral Performance |
|---|---|
| 4 points | No abnormal movement function |
| 3 points | Trembling of hind-limbs during tail suspension |
| 2 points | Anomalies of gait |
| 1 point | At least one hind-limb showing dragging. |
| 0 points | Placing mouse in a supine position, then failing to |
| | turn over to a prone position within 30 seconds |

### 2.4 Motor Function Assessment

### 2.4.1 Tail Suspension Test

The mice are suspended by their tails to observe hind-limb extension and body curvature, with scores given according to Table 2-2.

TABLE 2-2

| Score | Behavioral Performance |
|---|---|
| 2 points | Normal hind-limb extension and body curvature |
| 1 point | Partially normal hind-limb extension and body |
| | curvature |
| 0 points | Absence of hind-limb extension and body |
| | curvature |

### 2.4.1 Grip Strength Test

The grip strength of the mice is measured by using a grip strength meter. Each of the mice is gently placed on a grip plate and then pulled horizontally and slowly backward by the tail. When the mice completely release the grip plate, the grip strength meter automatically records a maximum grip strength value. This value is recorded, and each of the mice is tested three times. An average value is taken as the grip strength.

### 2.4.3 Mouse Treadmill Test

(1) Maximal exercise capacity test: At the start of the test, the speed is set at 12 meters per minute (m/min), then increased by 1 m/min every 3 minutes until the mouse fall off the treadmill. The speed, time, and distance at the moment before falling are recorded.

(2) Endurance test: A fixed speed is set at a challenging level for the mice (70% of the speed recorded in the maximal exercise capacity test, determined to be 15 m/min based on preliminary experimental data). The time and distance of the mice could maintain running until fatigue (falling off the treadmill) are recorded.

### 3.1 Post-Disease Weight Loss in hSOD1^{G93A} Transgenic Mice

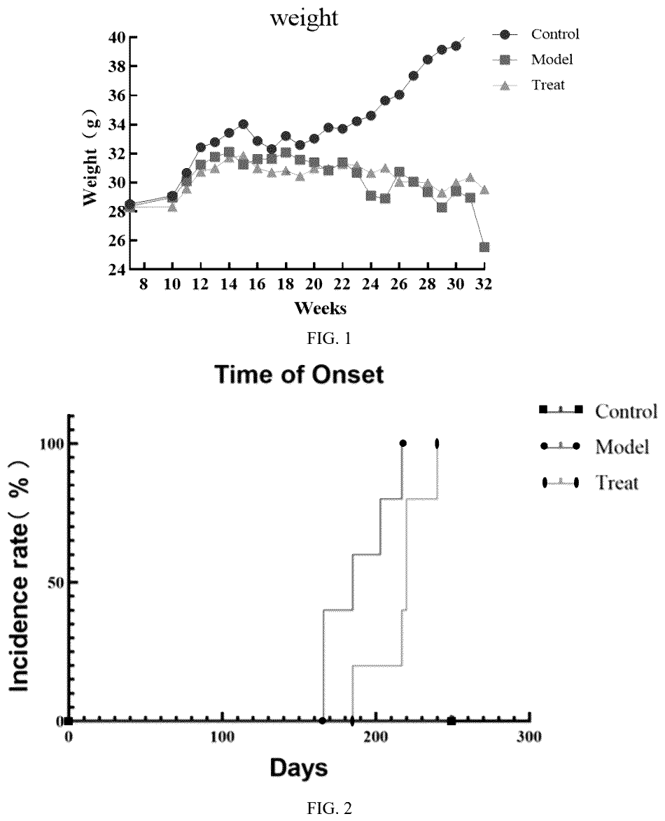

Body weight is measured in 7-week-old model mice. From week 7 to 14, the weight of mice in the control, model, and treated groups increased slowly with no significant differences. Starting from week 15, the weight of the model group shows a general downward trend, and the weight of the control group continues to rise slowly. Compared to the model group, the treated group does not show significant differences. However, compared to the control group, the hSOD1^{G93A} transgenic mice in the model group shows a highly significant weight difference (P<0.0001) (see FIG. 1).

### 3.2 Time of Onset and Survival Period Observation of Disease

3.2.1 Brucine Delays Disease Onset in hSOD1^{G93A} Transgenic Mouse

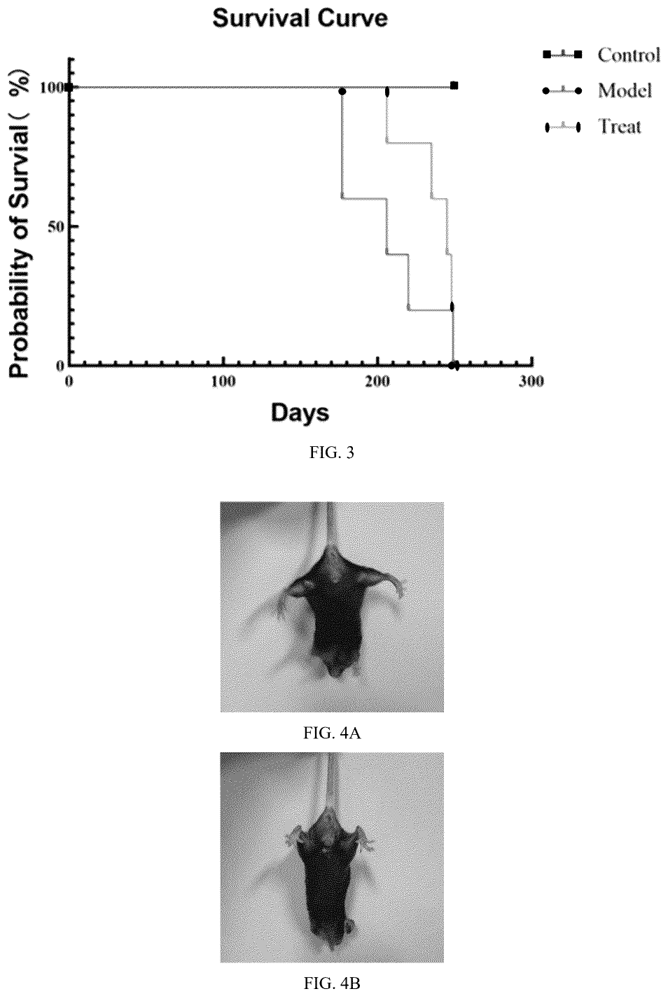

The average disease time of onset for mice in the model group is the 188^{th} day, compared to approximately 206^{th} day for the mice in the 10 mg/kg brucine-treated group. The P-value between the model group and the treated group is 0.0328, indicating a statistically significant difference in disease time of onset (see FIG. 2).

3.2.2 Brucine Prolongs Survival Period in hSOD1^{G93A} Transgenic Mice

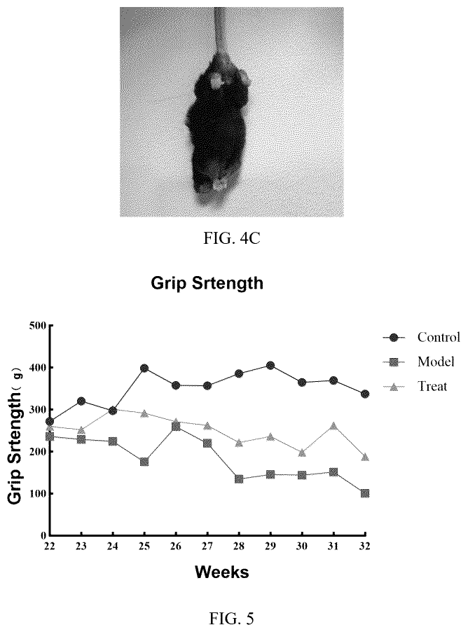

To date, four mice in the model group and one mouse in the treated group have died. The average survival period for mice in the model group is 200 days, while mice in the 10 mg/kg brucine-treated group have an average survival period of approximately 222 days. The P-value between the model group and the treated group is 0.0145, indicating a statistically significant difference in survival period (see FIG. 3).

### 3.3 Motor Function Assessment

3.3.1 Tail Suspension test

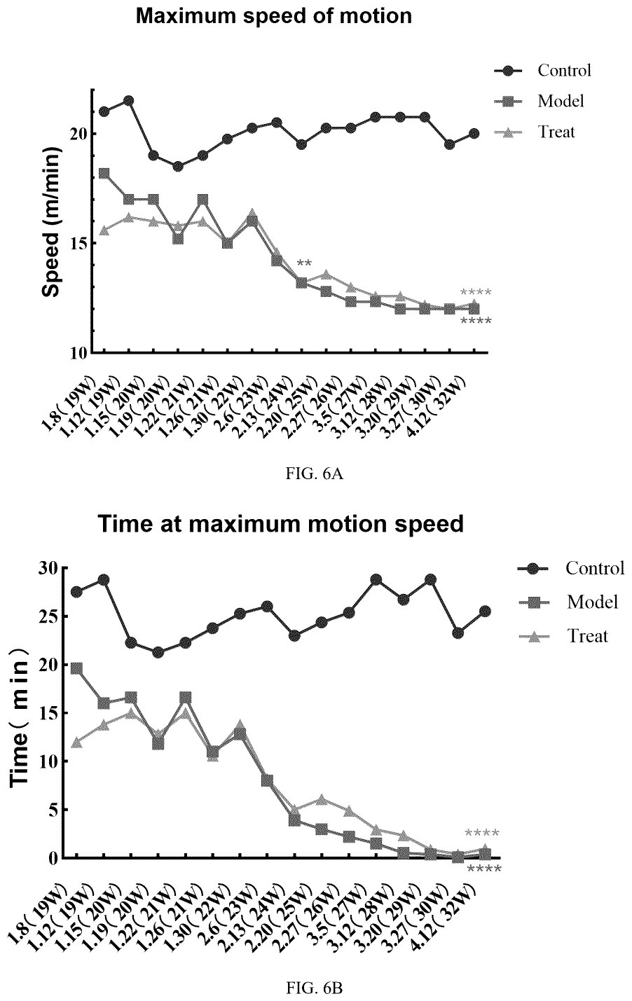

The hSOD1^{G93A} transgenic mice in model group, hind-limb extension ability gradually weakened with disease progression (FIGS. 4A-4C). At 6 months of age, mice treated with brucine show stronger hind-limb extension during the tail suspension test compared to the model group.

FIG. 4A shows hind-limb extension in hSOD1^{G93A} transgenic mice before disease onset; FIG. 4B shows hind-limb extension at disease onset; FIG. 4C shows hind-limb extension in late-stage disease.

### 3.3.2 Grip Strength Test

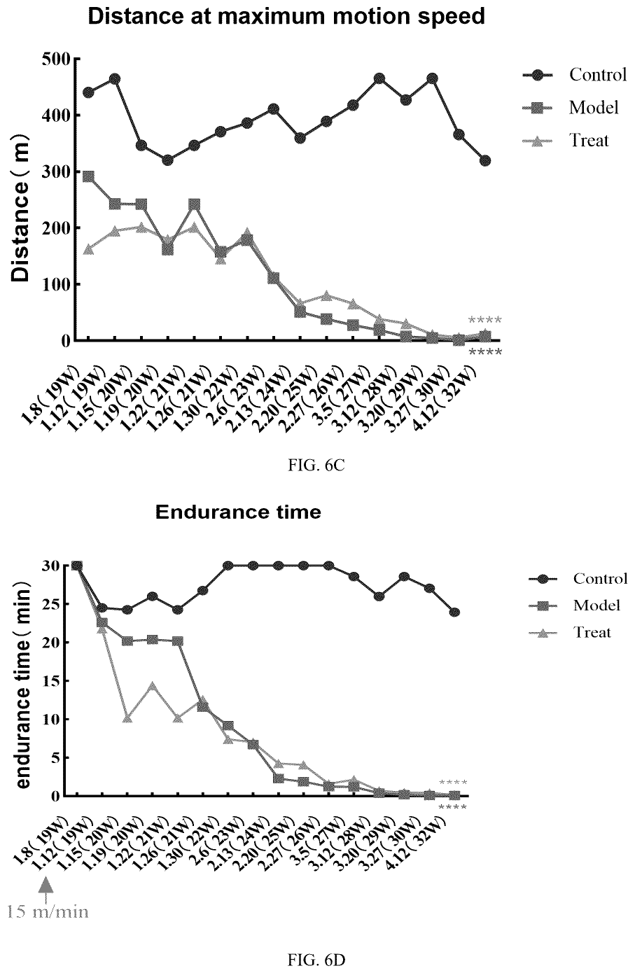

At the 22^{nd} week, no significant differences in grip strength are observed among the groups. At 25^{th} week, the model group shows significantly lower grip strength than the control group (P<0.0001), while the brucine-treated group has higher grip strength than the model group (P<0.05). For the hSOD1^{G93A} transgenic mice in the model group, grip strength shows a downward trend as the disease progressed (FIG. 5).

### 3.3.3 Mouse Treadmill Test

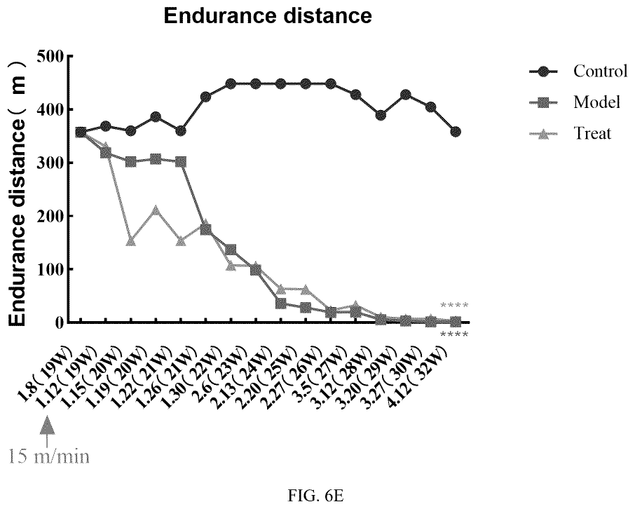

The hSOD1^{G93A} transgenic mice at 19^{th} week age are conducted with maximal exercise capacity test, the hSOD1^{G93A} transgenic mice in the model group show weaker maximal exercise capacity than the control group, which declines with disease progression. At 22^{nd} week (5.5 months), the maximal exercise capacity drops significantly, and at the 23^{rd} week (5.75 months), a marked difference from the control group is observed. In addition, the hSOD1^{G93A} transgenic mice at 19^{th} week age are conducted with an endurance test, the results are consistent with the maximal exercise capacity results, the endurance of hSOD1^{G93A} transgenic mice in the model group is weaker than the control group and declines with disease progression. At the 22^{nd} week (5.5 months), endurance time and distance decreases significantly, and at the 23^{rd} week (5.75 months), a marked difference from the control group is observed (FIG. 6).

FIGS. 6A-6C show maximum speed of motion, time at motion maximum speed, and distance at motion maximum speed of the hSOD1^{G93A} transgenic mice in the model group. FIGS. 6D-6E show the endurance training (i.e., endurance time and endurance distance) of the hSOD1^{G93A} transgenic mice in the model group.

In this study, the hSOD1^{G93A} transgenic mice are chosen as the ALS model mice. In survival experiments, time of onset and progressive symptoms of disease are recorded. The hSOD1^{G93A} transgenic mice typically start showing symptoms around the 23rd week age (the 161^{st} day). After the onset, hind-limb tremors occur during the tail suspension of the hSOD1^{G93A} transgenic mice. As the disease worsens, hind-limb weakness, gait abnormalities, and dragging movements appear. In late-stage disease, the hSOD1^{G93A} transgenic mice exhibit hind-limb paralysis, body atrophy, a curved spine, feeding difficulties, and rapid weight loss.

Weight or body mass index (BMI) decline is an independent prognostic factor for ALS. Mouse weights are recorded from 7 weeks of age. At this point, the hSOD1^{G93A} transgenic mice show no symptoms and have no significant weight difference from the control group. However, their weights begin to decrease around 12^{th} week age (3 months) and show a significant difference from the control group by the 21^{st} week (5.25 months). The brucine-treated group showed no significant improvement in body weight compared to the model group.

The hSOD1^{G93A} transgenic mice typically develop the disease around 5.5 months of age. As the disease progresses, their motor function gradually declines, with a significant drop during the disease peak. Prophylactic administration of 10 mg/kg brucine to the mice one month before disease onset, then behavioral experimental data such as grip, tail suspension, and treadmill is analyzed, which shows that compared to the model group, treatment with 10 mg/kg brucine improves the grip of the ALS mice.

In summary, the dosage of 10 mg/kg brucine significantly delays disease onset, prolongs survival, and enhances grip strength in ALS mice.

The above embodiments are an explanation of the disclosure, not a limitation of the disclosure. Any simple modification of the disclosure falls within the scope of protection of the disclosure.

What is claimed is:

1. An application method of brucine, comprising:
treating amyotrophic lateral sclerosis (ALS) by using the brucine.

2. The application method of the brucine as claimed in claim 1, wherein the ALS is a progressive, fatal neurodegenerative disease affecting motor neurons.

3. The application method of the brucine as claimed in claim 1, wherein the ALS is a predominant subtype of motor neuron disease (MND) and accounts for 80% to 90% of MND cases.

4. The application method of the brucine as claimed in claim 1, wherein the ALS is a mammalian ALS.

5. The application method of the brucine as claimed in claim 1, wherein the treating ALS by using the brucine comprises:
delaying a time of onset of the ALS and prolonging survival time of ALS patients.

6. The application method of the brucine as claimed in claim 1, wherein the treating ALS by using the brucine comprises:
delaying a weakening of limb extension ability.

7. The application method of the brucine as claimed in claim 1, wherein the treating ALS by using the brucine comprises:
improving limb grip strength.

8. The application method of the brucine as claimed in claim 1, wherein a dosage form of the brucine is one selected from the group consisting of tablets, capsules, granules, powders, pills, lozenges, dustpowder, solutions, syrups, suspensions, emulsions, and elixirs.

9. The application method of the brucine as claimed in claim 1, wherein the treating ALS by using the brucine comprises:
simultaneously administering the brucine and other drugs for treating the ALS.
